(19) United States
(12) Patent Application Publication
(10) Pub. No.: US 2019/0175680 A1
(43) Pub. Date: Jun. 13, 2019

(54) COMPOSITIONS COMPRISING NANOPARTICLES DERIVED FROM WHOLE FRUIT

(71) Applicant: Nanosmart Fruit, LLC, Traverse City, MI (US)

(72) Inventor: Robert Lee UNDERWOOD, Traverse City, MI (US)

(21) Appl. No.: 16/301,017

(22) PCT Filed: Mar. 22, 2017
(86) PCT No.: PCT/US2017/033733

§ 371 Date: Nov. 13, 2018

(51) Int. Cl. A61K 36/736; A61K 9/16; A61K 8/9789; A61K 31/7008; A61K 31/737; A61K 31/122; A61K 9/127; A61K 9/107; A61Q 5/02; A61Q 19/10; A61Q 19/00; A23L 19/00

(52) U.S. Cl. CPC A61K 36/736; A61K 9/16; A61K 8/9789; A61K 31/7008; A61K 31/737; A61K 31/122; A23V 2002/00; A61K 9/107; A61Q 5/02; A61Q 19/10; A61Q 19/00; A23L 19/01; A61K 9/127

(57) ABSTRACT

Nanoparticles may be derived from whole fruit. The nanoparticles may have a particles size less than about 1000 nanometers. The compositions may be used in nutraceutical products, dietary supplements, added to food or drinks, and combinations thereof.

### TECHNICAL FIELD

The present invention relates to compositions comprising nanoparticles derived from whole fruit having particles in a nano-range where the compositions are ingestable and suitable for topical application to skin and/or hair for mammals, such as humans or other animals.

### BACKGROUND

Many plant-derived compounds may impart important positive pharmacological or “nutraceutical/phytoceutical” traits to foods by way of their abilities to serve as antioxidants by maintaining low levels of reactive oxygen intermediates, as anti-inflammatory agents by inhibiting prostaglandin synthesis, or as inhibitors of enzymes involved in cell proliferation. These activities may be important in ameliorating chronic diseases including cancer, arthritis, cardiovascular disease, Alzheimer's disease, diabetes, and combinations thereof. Thus, with natural products, the dietary supplement/food industry and neutraceutical/phytoceutical companies have the opportunity to employ compounds which can offer significant health benefits to a consumer.

Some fruits are known to be high in anti-oxidants and have proven effective in improving health. Blueberries, pomegranates, plums, cranberries, black chokeberries (sanbucus nigra), black elderberries, and cherries are all relatively high in anti-oxidants. Consumption of cherries, and products derived from whole cherries, are known to provide a variety of additional health benefits. Tart cherries (prunus cerasus), especially the Montmorency, Morello, and Balaton varieties, are a good source of bioflavonoids, in addition to anti-oxidants.

Specifically, cherries are thought to have beneficial health properties, such as decreasing symptoms of arthritic pain, gout, diabetes, Alzheimer's disease, insomnia, hormone imbalance, and the like. These beneficial effects may be partially associated with the abundance of anthocyanins, i.e. the glycosides of cyanidin. Colorants like anthocyanins have been regarded as the index of quality in tart cherries. Most importantly, studies show that anthocyanins such as cyanidin-3-glucoside have strong antioxidant activities. Flavonoids are being investigated as anti-inflammatory substances as well as their structural features for cyclooxygenase (COX) activity. Melatonins are also present in cherries, which help with regulation of sleep patterns and balancing hormones.

There is a need for better whole fruit products having an improved bioavailability to be used or consumed by mammals for improving well-being of a human or other animal, such as a dog, a horse, a cow, and the like in a non-limiting embodiment.

### SUMMARY OF THE INVENTION

According to one non-limiting embodiment, a composition may include nanoparticles derived from whole fruit where the nanoparticles have a particle size less than about 1000 nanometers.

### DETAILED DESCRIPTION

It has been discovered that a composition including nanoparticles derived from whole fruit where the particles have a particle size less than about 1000 nanometers may improve the bioavailability of whole fruit products when consumed or used by mammals, such as humans, dogs, cows, horses, and the like. Such whole fruit products may be or include, but are not limited to nutraceuticals, dietary supplements, additives to food or drinks, and other related topical products (e.g. for skin and/or hair). “Mammal” as used herein is defined to be a warm-blooded animal having fur or hair.

The composition(s) may improve digestion, retard the aging process, reduce systemic inflammation (e.g. arthritis and/or gout), protect against certain types of cancer and heart disease, assist in stabilizing and balancing hormones, neutralize free radicals, reduce short-term memory loss, aid in combating stress, reduce high strain and fatigue, reduce LDL cholesterol, improve natural sleep patterns, maintain a long and stable shelf life, improve the ability to heal burns to the skin, and combinations thereof, all without disruptive side effects.

“Whole fruit” is used herein to refer to fruits that are relatively unprocessed and/or unrefined before being consumed or used. Said differently, ‘whole fruit nanoparticles’, or ‘nanoparticles’, refers to particles derived from physically processing (e.g. pulverizing, mashing, pureeing, and combinations thereof) at least one whole fruit into its relevant whole fruit nanoparticles. “Whole fruit” is used herein to refer to the edible portion of a fruit and does not include leaves, sterns, soil, etc.

In a non-limiting embodiment, the whole fruit may become whole fruit nanoparticles by pulverizing the whole fruit, i.e. by grinding, crushing, milling, mashing, and combinations thereof the whole fruit until the whole fruit becomes a nanoparticle size. When pulverizing the whole fruit, the whole fruit first becomes a puree, which includes skin, juice, pulp, and/or other parts of the whole fruit that may be ground into nanoparticles. The puree is then sieved through a wire mesh to remove any larger unwanted particles, such as seeds, pits, stems, debris, and combinations thereof. The puree comprising the nanoparticles may be heated to a temperature below the boiling point of the puree for purposes of killing any microorganisms (i.e. bacteria, mold, fungus, etc.), but the temperature is low enough to maintain the structure of the antioxidants, anthocyanins, bioflavonoids, phenolics, melatonins, and combinations thereof,

In yet another non-limiting embodiment, the fluid (i.e. juice, water, etc.) may be removed from the puree to form a whole fruit paste. Again, the whole fruit puree may be heated to a temperature where the water may be removed as steam without boiling the whole fruit puree. The paste may have a substantially increased viscosity compared to that of the whole fruit puree, such as but not limited to a viscosity ranging from about 50,000 cps (about 50 pascal seconds) independently to about 300,000 cps (about 300 pascal seconds), alternatively from about 100,000 cps (about 100 pascal seconds) independently to about 250,000 cps (250 pascal seconds), or from about 150,000 cps (150 pascal seconds) independently to about 200,000 cps (200 pascal seconds) in another non-limiting embodiment. Said differently, the whole fruit paste may have a viscosity similar to that of peanut butter in a non-limiting embodiment.

The whole fruit puree and/or the whole fruit paste may be incorporated and/or formed as a nutraceutical, a food, a drink, a dietary supplement, a topical agent, and combinations thereof. The nanoparticles within the composition may be disposed in a form, such as but not limited to, a solid, a powder, a paste, a concentrate, a tablet, a gel capsule, an enteric coated capsule, a cream, a massage oil, a lotion, a gel (e.g. where the composition further includes glycerin), a shampoo, a soap, a spray, a single-serve ‘packet’, and combinations thereof. In addition, a water-based fluid and/or an oil-based fluid may be further incorporated thereinto depending on the intended use of the end-product.

In a non-limiting embodiment, the particle size of the nanoparticles may range from about 101 nm independently to about 999 nm, or from about 200 nm independently to about 800 nm, from about 350 nm independently to about 650 nm, or from about 400 nm independently to about 550 nm. As used herein with respect to a range, “independently” means that any threshold may be used together with another threshold to give a suitable alternative range, e.g. about 200 nm independently to about 350 nm is also considered a suitable alternative range.

In a non-limiting embodiment, the whole fruit nanoparticles may be or include particles from at least one fruit, such as but not limited to blackberries, strawberries, cherries, blueberries (wild and/or cultivated), pomegranates, raspberries, plums, cranberries, black chokeberries, black elderberries, and combinations thereof. Alternatively, the nanoparticles may include cherry particles from at least one of Balaton cherries, Bing cherries, Lambert cherries, Maraschino cherries, Morello cherries, Ranier cherries, Montmorency cherries, Van cherries, Chelan cherries, Lapin cherries, Sweetheart cherries, Skeena cherries, Staccato cherries, wild cherries, sweet cherries, sour cherries, and combinations thereof.

The composition may be incorporated and/or formed as a nutraceutical, a food, a drink, a dietary supplement, a topical agent, and combinations thereof. The nanoparticles within the composition may be disposed in a form, such as but not limited to, a solid, a powder, a paste, a concentrate, a tablet, a gel capsule, an enteric-coated capsule, a cream, a massage oil, a lotion, a gel, a shampoo, a soap, a spray, and combinations thereof.

The nanoparticles may be incorporated as a freeze-dried powder manufactured by a vacuum freeze drying process. A non-limiting example of a commercial source of freeze dried red tart cherry powder is available from Crystals International, Inc., 600 West Dr. M. L. King, Jr. Blvd., Plant City, Fla., which includes 70% red tart cherry juice concentrate. Whole fruit powder may also be made from a spray dry process, a drum dry process, a pan dry process, and combinations thereof. Other acceptable forms of whole fruit nanoparticles may be or include fruit purees and/or fruit pastes having particles from at least one or more whole fruits.

In another non-limiting form, nanoparticles may be reduced to a form consumed by mammals, e.g. humans and/or other animals, such as but not limited to an oral administration of a gelatin capsule comprising the composition. Other non-limiting examples may be or include a powdered form of nanoparticles to be added to a drink or sprinkled on food, a powdered form of nanoparticles disposed in a capsule, a dried form of the composition pressed into a tablet, a puree including the nanoparticles and at least one natural fruit fluid, and the like.

When used as a nutraceutical, dietary supplement, food additive, drink additive, and combinations thereof, the composition may include from about 100 mg independently to about 3000 mg of nanoparticles, alternatively from about 500 mg independently to about 2000 mg, or from about 1000 mg independently to about 1500 mg in another non-limiting embodiment.

In addition or in the alternative, anthocyanins, bioflavonoids, phenolics, and combinations thereof may be isolated from the whole fruit particles, and the isolated anthocyanins, bioflavonoids, phenolics, melatonins, and combinations thereof may be combined with the nanoparticles to be provided as a natural nutraceutical, phytoceutical, or dietary supplement. In a non-limiting embodiment, one hundred (100) cherries provide about 10 independently to about 100 mg of anthocyanins and bioflavonoids. In another non-limiting embodiment, one hundred cherries may provide about 1 independently to about 50 mg of phenolics.

In yet another non-limiting embodiment, a composition agent may be added or included in the composition, such as but not limited to, an emulsifier, a thickener, a dispersant, a diluent, a preservative, a flavoring, and combinations thereof. Non-limiting examples of composition agents may be or include, but are not limited to, dried fruit pulp, any edible starch containing material, protein, and combinations thereof, e.g. dry milk, flour, sugar, lecithin, maltodextrin, soybean meal, salt, pepper, spices, glycerin, bee's wax and combinations thereof. The composition agents may improve solubility and/or viscosity characteristics of the composition.

The composition may include at least one additional nutritional component, such as but not limited to, glucosamine, chondroitin sulfate, coenzyme Q10, a vitamin, a mineral, an herb, a spice, and combinations thereof. The addition of coenzyme Q10 may aid in preventing heart disease, brain diseases, periodontal diseases, and the like. Non-limiting examples of vitamins may be or include, but are not limited to vitamin A, vitamin B₃, vitamin B₅, vitamin B₆, vitamin B₁₂, vitamin C, vitamin D₃, vitamin E, vitamin K₁, vitamin K₂, and combinations thereof. Non-limiting examples of minerals may be or include boron, calcium, iron, magnesium, selenium, vanadium, zinc, and combinations thereof. Non-limiting examples of spices may be or include turmeric, sage, rosemary, ginger, cinnamon, saffron, parsley, and combinations thereof.

In addition or in the alternative, the composition may include a fluid where the nanoparticles are substantially equally dispersed in the fluid. When the nanoparticles are combined with a fluid to form a fluid composition, the nano-size of the particles may decrease the probability of the nanoparticles from falling out of a suspension or emulsion as compared to the same micron-sized particles disposed, mixed, or dispersed in a fluid. The nano-size of the particles ensures the same amount of the nanoparticles is disposed in each portion of the fluid composition. Said differently, the nano-sizing of the particles allows for a more ‘equal distribution’ of the nanoparticles when the particles are disposed, mixed, emulsified, and/or encapsulated in a fluid.

In a non-limiting embodiment, the fluid may be or include a water-based fluid, an oil-based fluid, and combinations thereof. In water-based fluids, the processed whole fruit may be suspended in water, brine, a fruit juice/concentrate, or another fluid where water is the primary solvent to form a solution with the nanoparticles. An oil may be emulsified in the water-based fluid, but water is a continuous phase within a ‘water-based fluid’.

Fruit concentrate may be made according to procedures available to those skilled in the art, such as but not limited to freezing pitted whole fruit (e.g. cherries) and then partially thawing the frozen pitted whole fruit to allow for juice to be pressed from the whole fruit. In addition or in the alternative, the whole fruit may be processed into microsized and/or nanosized particles and then centrifuged to a concentration ranging from about 45 brix independently to about 68 brix, or from about 50 brix independently to about 60 brix. The term “brix” is used as a unit of measure of sucrose (the percent of sugar in 100 g of cane sugar solution). In a non-limiting embodiment, it takes about 80 (about 36.3 kg) to about 90 lbs. (about 40.8 kg) of cherries to render 1 gallon (about 3.79 liters) of cherry concentrate. In yet another non-limiting embodiment, at least a portion of sugar may be removed from the concentrate and/or composition, regardless of whether the composition is dry or in fluid form.

Oil-based fluids are the opposite or inverse, i.e. where the whole fruit nanoparticles are suspended in oil in a non-limiting embodiment. A water-based fluid may be emulsified in at least one oil, such that the oil(s) are the continuous phase within the oil-based fluid. In a non-limiting embodiment, the oil-based fluid may include at least one oil, such as but not limited to emu oil, coconut oil, fish oil, olive oil, flaxseed oil, and combinations thereof. The oil(s) may be refined for purposes of removing impurities, discoloration, etc. A non-limiting process for refining oil is commonly termed “cold pressing” in which the temperature of the oil is not raised above about 50 C, to keep the oil stable. In another non-limiting embodiment, the oil may be an organic oil as defined by the United States Department of Agriculture (USDA).

In a non-limiting form, at least one type of oil and whole fruit particles (e.g. cherries) may be combined to form a cream, an oil, or a lotion for topical use on skin of a mammal, e.g. humans or other mammal animals. All of the natural health benefits of the whole fruit nanoparticles may be transported subcutaneously by the oil's ability to penetrate the skin. In yet another non-limiting embodiment, the nanoparticles may be encapsulated in at least one of a liposome, an emulsion, and combinations thereof.

In a non-limiting embodiment, the composition may include a fluid having one or more aliquots. Each aliquot may include an amount of nanoparticles ranging from about 100 mg independently to about 3000 mg of nanoparticles, alternatively from about 500 mg independently to about 2000 mg, or from about 1000 mg independently to about 1500 mg in another non-limiting embodiment.

In a non-limiting embodiment, a gel composition may be or include a whole fruit paste, a whole fruit puree, a composition agent, and an optional flavor enhancer. The gel may include the whole fruit paste in an amount ranging from about 45 independently to about 65% by weight, alternatively, from about 50% independently to about 60%. The gel may include the whole fruit puree in an amount ranging from about 20% independently to about 30% by weight, alternatively from about 22% independently to about 27%. The gel may include the composition agent in an amount ranging from about 5% independently to about 20%, alternatively from about 10% independently to about 16%. The whole fruit paste and/or whole fruit puree may include nanoparticles derived from one or more of the whole fruits and/or whole cherries as mentioned herein. The composition agent may be or include dry milk, flour, sugar, lecithin, maltodextrin, soybean meal, salt, pepper, spices, glycerin, and combinations thereof. In an alternative embodiment, the gel may include chocolate (sugar-free or regular chocolate) or another flavor enhancer in an amount ranging from about 1% independently to about 20%, or from about 12% independently to about 16% in another non-limiting embodiment.

In another non-limiting embodiment, a gel composition may be or include a whole fruit paste, a whole fruit concentrate, a composition agent, and an optional flavor enhancer. The gel may include the whole fruit paste in an amount ranging from about 45% independently to about 65% by weight, alternatively, from about 50% independently to about 60%. The gel may include the whole fruit concentrate in an amount ranging from about 15% independently to about 35%, or from about 20% independently to about 30% in another non-limiting embodiment. The gel may include the composition agent in an amount ranging from about 5% independently to about 20%, alternatively from about 10% independently to about 16%. The whole fruit paste and/or the whole fruit concentrate may include nanoparticles derived from one or more of the whole fruits and/or whole cherries as mentioned herein. The composition agent may be or include dry milk, flour, sugar, lecithin, maltodextrin, soybean meal, salt, pepper, spices, glycerin, and combinations thereof. In an alternative embodiment, the gel may include chocolate (sugar-free or regular chocolate) or another flavor enhancer in an amount ranging from about 1% independently to about 20%, or from about 5% independently to about 15% in another non-limiting embodiment.

In a non-limiting embodiment, a softgel capsule composition may be or include an oil-based fluid, a composition agent, a whole fruit powder, a whole fruit paste, and combinations thereof. The softgel capsule composition may include the oil-based fluid in an amount ranging from about 40% independently to about 60% by weight, or from about 45% independently to about 55% in another non-limiting embodiment. The softgel capsule composition may include the composition agent in an amount ranging from about 1% independently to about 5%, or from about 2% independently to about 4% in another non-limiting embodiment. The softgel capsule composition may include a whole fruit powder in an amount ranging from about 5% independently to about 15%, or from about 8% independently to about 12%. The softgel capsule composition may include the whole fruit paste in an amount ranging from about 30% independently to about 60%, or from about 40% independently to about 50% in another non-limiting embodiment. The softgel capsule composition may include the composition agent in an amount ranging from about 1% independently to about 10%, alternatively from about 2% independently to about 5% in another non-limiting embodiment. The whole fruit powder and/or whole fruit paste may include nanoparticles derived from one or more of the whole fruits and/or whole cherries as mentioned herein. The composition agent may be or include dry milk, flour, sugar, lecithin, maltodextrin, soybean meal, salt, pepper, spices, glycerin, and combinations thereof.

In another non-limiting embodiment, a whole fruit “shot” composition may include at least one whole fruit concentrate and/or whole fruit puree, and at least one whole fruit paste. The whole fruit shot composition may include at least one whole fruit concentrate in an amount ranging from about 50% independently to about 80% by weight, or from about 60% independently to about 75% in another non-limiting embodiment. The whole fruit composition may include at least one whole fruit paste in an amount ranging from about 20% independently to about 40%, or from about 25% independently to about 35% in another non-limiting embodiment. The whole fruit concentrate and/or whole fruit paste may include nanoparticles derived from one or more of the whole fruits and/or whole cherries as mentioned herein. A “shot” has used herein refers to the composition comprising a liquid in an amount ranging from about 1 oz (about 3 centiliters) independently to about 4 oz (about 11.8 cL), or from about 2 oz (about 6 cL) independently to about 3 oz (about 8.9 cL), or about 2.5 oz (about 7.4 cL) in another non-limiting embodiment.

In yet another non-limiting embodiment, the whole fruit shot composition may be incorporated into a water-based fluid where the whole fruit shot to water-based fluid ranges from about 1 parts whole fruit shot independently to about 1 parts water-based fluid, or from about 1 parts whole fruit shot independently to about 2 parts water-based fluid in another non-limiting embodiment; alternatively, the ratio of whole fruit shot ranges from about 2 parts whole fruit shot to about 3 parts water-based fluid.

In yet another non-limiting embodiment, a single-serve packet or “stick” may include the whole fruit puree and/or the whole fruit paste in a serving size less than 2 ounces (about 6 cL) in a non-limiting embodiment; the single-serve packet may optionally include glycerin or another component to alter the consistency and/or flavor of the single-serve packet. If whole fruit puree is not included in the single-serve packet, a water-based fluid and/or an oil-based fluid may be incorporated thereinto to increase the fluidity of the whole fruit paste.

While the invention has been specifically described in connection with certain specific embodiments thereof, it is to be understood that this is by way of illustration and not of limitation, and the scope of the appended claims should be construed as broadly as the prior art will permit.

The present invention may suitably comprise, consist or consist essentially of the elements disclosed and may be practiced in the absence of an element not disclosed. For instance, the composition may consist of or consist essentially of nanoparticles derived from whole fruit, wherein the nanoparticles have a particle size less than about 1000 nanometers.

The words “comprising” and “comprises” as used throughout the claims, are to be interpreted to mean “including but not limited to” and “includes but not limited to”, respectively.

In the foregoing specification, the invention has been described with reference to specific embodiments thereof, and has been described as effective in providing nanoparticle compositions derived from whole fruit. However, it will be evident that various modifications and changes can be made thereto without departing from the broader scope of the invention as set forth in the appended claims. Accordingly, the specification is to be regarded in an illustrative rather than a restrictive sense. For example, specific whole fruits, specific cherries, oil-based fluids, water-based fluids, composition agents, nutritional components, and the like falling within the claimed parameters, but not specifically identified or tried in a particular composition or method, are expected to be within the scope of this invention.

1. A composition comprising whole fruit nanoparticles, wherein the nanoparticles have a particle size less than 1000 nanometers.

2. The composition of claim 1, wherein the at least one whole fruit is selected from the group consisting of cherries, blueberries, pomegranates, raspberries, plums, cranberries, black chokeberries, black elderberries, and combinations thereof.

3. The composition of claim 2, wherein the cherries are selected from the group consisting of Balaton cherries, Bing cherries, Lambert cherries, Maraschino cherries, Morello cherries, Ranier cherries, Montmorency cherries, Van cherries, Chelan cherries, Lapin cherries, Sweetheart cherries, Skeena cherries, Staccato cherries, wild cherries, sweet cherries, sour cherries, tart cherries, and combinations thereof.

4. The composition of claim 1, wherein the nanoparticles are in a form selected from at least one of a solid, a powder, a paste, a puree, a tablet, a gel capsule, an enteric coated pill, and combinations thereof.

5. The composition of claim 1, wherein the composition comprises one or more portions, and wherein each portion comprises the nanoparticles in an amount ranging from 100 mg to 3000 mg.

6. The composition of claim 1, wherein the nanoparticles comprise a particle ranging from 101 nm to 999 nm.

7. The composition of claim 1, wherein the composition further comprises at least one fluid selected from the group consisting of a water-based fluid, an oil-based fluid, and combinations thereof.

8. The composition of claim 7, wherein the oil-based fluid comprises at least one oil selected from the group consisting of emu oil, coconut oil, fish oil, olive oil, flaxseed oil, and combinations thereof.

9. The composition of claim 7, wherein the nanoparticles are substantially equally dispersed in the fluid.

10. The composition of claim 7, wherein the nanoparticles are at least one of disposed, mixed, emulsified, dispersed, and/or combinations thereof in the fluid.

11. The composition of claim 1, wherein at least a portion of sugar has been removed from the composition.

12. The composition of claim 7, wherein the composition is in a form selected from the group consisting of a suspension, a concentrate, a cream, a massage oil, a lotion, a gel, a shampoo, a soap, a spray, an emulsion, and combinations thereof.

13. The composition of claim 1, further comprising at least one agent selected from the group consisting of an emulsifier, a thickener, a dispersant, a diluent, a thickener, a preservative, a flavoring, and combinations thereof.

14. The composition of claim 1, wherein the composition further comprises at least one nutritional component selected from the group consisting of glucosamine, chondroitin sulfate, coenzyme Q10, a vitamin, a mineral, an herb, a spice, and combinations thereof.

15. The composition of claim 1, wherein the nanoparticles are encapsulated in at least one of a liposome, an emulsion, and combinations thereof.
